(19) United States
(12) Patent Application Publication
(10) Pub. No.: US 2007/0276049 A1
(43) Pub. Date: Nov. 29, 2007

(54) Regulation of Cell Membrane-Mediated Effects

(76) Inventors: Patrick M. Schlievert, Edina, MN (US); Marnie L. Peterson, Golden Valley, MN (US)

Correspondence Address: FISH & RICHARDSON P.C., P.O. BOX 1022, MINNEAPOLIS, MN, 55440-1022, US

(21) Appl. No.: 10/579,108

(22) PCT Filed: Nov. 10, 2004
(86) PCT No.: PCT/US04/37466

§ 371 Date: Jun. 18, 2007

(30) Foreign Application Priority Data
Nov. 11, 2003 (US) 60/519339
Aug. 20, 2004 (US) 60/603,117

(51) Int. Cl. A61K 31/12; A61K 31/045

(52) U.S. Cl. 514/738; 514/675

(57) ABSTRACT

The invention features methods of inhibiting cell membrane-mediated effects of infectious microorganisms and of a variety of factors on vertebrate cells with one or more of a family of glycerol-based compounds. The cell membrane-mediated s effects are those resulting in, for example, infection of the vertebrate cells by relevant infectious microorganisms and inflammatory responses to infectious microorganisms or factors.

### STATEMENT REGARDING FEDERALLY SPONSORED RESEARCH OR DEVELOPMENT

Some of the research described in this application was supported by grant Nos. HL36611 and AI57164 from the National Heart, Lung, and Blood Institute and the National Institute of Allergy and Infectious Diseases, respectively, of the National Institutes of Health. Thus the government has certain rights in the invention.

### TECHNICAL FIELD

This invention relates to pathologic conditions that are associated with cell membrane-mediated effects in vertebrate subjects, and more particularly to the regulation of such effects.

### BACKGROUND

Due to the human, social, and economic devastation that continues to be wreaked by infectious pathogens (and other agents, such as pathogenic toxins and inflammatory factors), which exert their pathogenic activity via cell membrane-mediated effects on host cells, it remains imperative that effective new therapeutic and/or prophylactic compounds that are both inexpensive and logistically simple to deliver to appropriate subjects continue to be developed.

### SUMMARY

The invention is based in part upon the inventors' discovery that glycerol monolaurate (GML) inhibits pathogenic cell membrane-mediated effects on vertebrate cells of infectious microorganisms and/or of factors produced by infectious microorganisms.

For example, the inventors have observed that infectious microorganisms and/or microbial factors (e.g., exotoxins, endotoxin, such as lipopolysaccharide, or superantigens) have cell membrane-mediated effects on mammalian cells. Such cell membrane-mediated effects include, for example, enhancement of lymphocyte proliferation, infection, and/or activation of inflammatory responses, of epithelial cells, and lysis of red blood cells (RBCs). The inventors have found, moreover, that GML ameliorates or abrogates these cell membrane-mediated effects. In light of these observations, GML and related molecules are likely to be effective therapeutic, and/or prophylactic, agents against pathologic conditions that are associated with such cell membrane-mediated effects such as those described herein. Furthermore, in view of the observation that GML exerted an inhibitory effect on infection by an obligate intracellular bacterium, Chlamydia trachomatis, and a virus, human immunodeficiency virus (HIV) 1 (HIV-1), it is likely that it and related compounds will be effective in inhibiting a cell membrane-mediated effect on a vertebrate cell caused by contact of the vertebrate cell with other viruses and obligate intracellular pathogens. GML and related compounds are simple and inexpensive to produce in large amounts, transport, and administer to relevant subjects. Moreover, in that the targets of such drugs are not infectious microorganisms themselves, the development of drug-resistance by relevant infectious organisms will not be a disadvantage of regimens using the drugs.

More specifically, the invention provides a method of inhibiting a cell membrane-mediated effect. The method includes: (a) identifying a vertebrate subject as having been, as likely to have been, or likely to be, exposed to an infectious microorganism, or a microbial factor associated with an infectious microorganism, the infectious microorganism having a cell membrane-mediated effect on a vertebrate cell, the cell membrane-mediated effect being associated with a pathological condition of the vertebrate subject; and (b) administering to the subject an isolated glycerol-based compound that (i) inhibits the cell membrane-mediated effect and (ii) is a glycerol-based compound containing a structure that is:

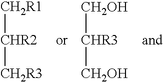

R1 can be: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁ CH₃; O(CH₂)₁₂CH₃; or O(CH₂)₁₃CH₃,

R2 can be: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃, and

R3 can be: CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃.

The vertebrate cell can be, for example, an epithelial cell, an RBC, or a lymphocyte, e.g., a T cell or a B cell. The infectious microorganism can be a bacterium, e.g., a Staphlylococcus (such as Staphylococcus aureus); a Neisseria (such as Neisseria gonorrheae); a Streptococcus (such as Streptococcus pyogenes); Chlamydia trachomatis; Gardnerella vaginalis; Haemophilus ducreyi; a bacillus (such as Bacillus anthracis); a Clostridium (such as Clostridium perfringens); Haemophilus ducreyi; and Treponena pallidum. The infectious microorganism can also be a virus, e.g., HIV-1, HIV-2, Herpes Simplex Virus (HSV), or Human Papilloma Virus (HPV). Moreover the infectious microorganism can be a protozoan such as Trichomonas vaginalis or a fungus such as Candida albicans. The subject can be any vertebrate (e.g., mammalian) subject recited herein, e.g., a human.

The invention embodies another method of inhibiting a cell membrane-mediated effect. The method includes: (a) (i) identifying a vertebrate subject as likely to have, or likely to develop, a pathological condition that is associated with a cell membrane-mediated effect on a vertebrate cell in the subject of a factor to which the subject is exposed, or (ii) identifying a vertebrate subject as having been, as likely to have been, or likely to be exposed to a factor that has cell-mediated effect on a vertebrate cell, wherein the cell-mediated effect can result in a pathologic condition in the vertebrate subject; and (b) administering to the subject an isolated glycerol-based compound, that can be any of these listed above, that inhibits the cell membrane-mediated effect on the vertebrate cell by the factor. Factors that can cause cell membrane-mediated effects on vertebrate cells include a wide variety of naturally occurring and synthetic substances including, without limitation, small molecules and biological molecules such as proteins, nucleic acids, carbohydrates, and lipids, and can be any of the microbial and non-microbial substances recited herein. The vertebrate cell and the subject can be any vertebrate cell and any vertebrate subject, respectively, recited herein. The pathological condition can be, for example, psoriasis, atopic dermatitis, or a skin papule or pustule, toxic shock syndrome (TSS), pneumonia, bacteremia in association with cutaneous infection (e.g., cellulitis, erysipelas, or infection of a surgical or nonsurgical wound), deep soft tissue infection (e.g., myositis or necrotizing fasciitis), meningitis, peritonitis, osteomyelitis, septic arthritis, postpartum sepsis (e.g., puerperal fever), neonatal sepsis, all forms of food poisoning (e.g., Salmonellosis, staphylococcal enterotoxin-mediated food poisoning, Escherichia coli travelers' diarrhea, cholera, Shigellosis, and botulism), and viral infection.

In another aspect, the invention provides an in vitro method of inhibiting a cell membrane-mediated effect. The method involves: (a) culturing a vertebrate cell with (i) an infectious microorganism that has a cell membrane-mediated effect on the vertebrate cell, or that elicits the production in the culture of a vertebrate mediator produced by a cell in the culture, that has the cell membrane-mediated effect on the vertebrate cell; or (ii) a microbial factor that causes a cell membrane-mediated effect on the vertebrate cell; and (b) before, simultaneous with, or after step (a), contacting the vertebrate cell with an isolated glycerol-based compound that can be any of those listed above. The vertebrate mediator can be any of the inflammatory or immunostimulatory mediators recited herein and can be produced by the vertebrate cell referred to above or any other vertebrate cell in the culture. The glycerol-based compound inhibits the cell membrane-mediated effect on the vertebrate cell by the infectious microorganism, the vertebrate mediator that is elicited in the culture by the infectious microorganism, and/or the microbial factor. The vertebrate cell, microbial the factor, and the infectious microorganism can be any of those recited herein.

“Polypeptide” and “protein” are used interchangeably and mean any peptide-linked chain of amino acids, regardless of length or post-translational modification. Polypeptides for use in the invention include those with conservative substitutions. Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. In general, variant polypeptides with conservative substitutions will contain no more than 40 (e.g., no more than: 35; 30; 25; 20; 15; 13; 11; 10; nine; eight; seven; six; five; four; three; two; or one) conservative substitution(s). All that is required is that the variant polypeptides have at least 20% (e.g., at least: 30%; 40%; 50%; 60%; 70%; 80%; 90%; 95%; 98%; 99%; 99.5%; 99.8%; 99.9%; or 100% or more) of the activity of the wild-type polypeptide.

As used herein, a “microbial factor” is a factor: (a) made by an infectious microorganism (e.g., a bacterium); or (b) produced by a vertebrate cell infected by an infectious microorganism (e.g., a virus) but not by the same vertebrate cell if it is not infected with that infectious microorganism. Thus, a “microbial factor” can be, for example, a hemolysin produced by a bacterium, a virally encoded protein produced by a vertebrate cell infected with the relevant virus, or a product of a reaction in a vertebrate cell catalyzed by an enzyme encoded by an infectious microorganism harbored by the vertebrate cell. On the other hand, a “microbial factor” is not, for example, a wild-type factor (e.g., an inflammatory or immunostimulatory mediator) produced by, and encoded by genes of, a vertebrate cell infected with an infectious microorganism that the vertebrate cell has the ability to produce even if not infected with an infectious microorganism. Hence, a “microbial factor associated with an infectious microorganism” is a factor that can be produced by: (a) that infectious microorganism; or (b) a vertebrate cell infected with that infectious microorganism but not by a vertebrate cell that is not infected with any infectious microorganism.

The term “isolated compound” as used herein refers to a compound (e.g., GML) that either has no naturally-occurring counterpart or has been separated or purified from components which naturally accompany it, e.g., in tissues such as pancreas, liver, spleen, ovary, testis, muscle, joint tissue, neural tissue, gastrointestinal tissue or tumor tissue, or body fluids such as blood, serum, or urine. Typically, a naturally occurring biological compound is considered “isolated” when it is at least 70%, by dry weight, free from other naturally occurring organic molecules with which it is naturally associated. Preferably, a preparation of a compound for use in the invention is at least 80%, more preferably at least 90%, and most preferably at least 99%, by dry weight, that compound. The degree of isolation or purity can be measured by any appropriate method, e.g., column chromatography, polyacrylamide gel electrophoresis, or HPLC analysis. Since a compound (e.g., GML) that is chemically synthesized is, by its nature, separated from the components that naturally accompany it, the synthetic compound is by definition “isolated.”

Isolated compounds, and additional agents useful for the invention, can be obtained, for example, by: (i) extraction from a natural source (e.g., from tissues or bodily fluids); (ii) where the compound is a protein, by expression of a recombinant nucleic acid encoding the protein; or (iii) by standard chemical synthetic methods known to those in the art. A protein that is produced in a cellular system different from the source from which it naturally originates is “isolated,” because it will necessarily be free of components that naturally accompany it.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., inhibiting infection of cells, will be apparent from the following description and from the claims.

### DESCRIPTION OF DRAWINGS

FIG. 1 is a bar graph showing levels of cytokine (A) and chemokine (B) proteins in the supernatants of HVEC cultures after exposure to S. aureus Toxic Shock Syndrome Toxin-1 (TSST-1; 100 μg/ml) for 3 and 6 hours.

### DETAILED DESCRIPTION

The experiments of the inventors outlined in the Summary section indicate that GML inhibits cell membrane-mediated effects of a variety of bacteria and factors (e.g., microbial and non-microbial) on vertebrate cells. Examples are provided that illustrate the ability of GML to inhibit cell membrane-mediated effects and/or biological processes that are activated by cell membrane-mediated effects in vertebrate cells. For example, in human vaginal epithelial cells (HVECs), GML inhibited (i) infection by a variety of bacteria (e.g., S. aureus, N. gonorrhoeae, S. pyogenes, and C. trachomatis); and (ii) expression of genes (in terms of both mRNA and protein synthesis) encoding inflammatory mediators (e.g., chemokines and cytokines) in response to infectious microorganisms (e.g., S. aureus) or to exotoxins (e.g., S. aureus TSST-1). Moreover, GML inhibited infection of human T cells by HIV-1. GML also inhibited the lysis of RBCs by a variety of bacterial exotoxins (e.g., TSST-1, α-hemolysin, streptolysin O, and streptolysin S, and B. anthracis exotoxin). In addition, GML inhibited the proliferation of lymphocytes (e.g., T and B cells) after activation by the bacterial products TSST-1 and lipopolysaccharide (LPS). Thus, it seems that GML, and related compounds, can inhibit, or even ablate, vertebrate cell membrane-mediated effects of any of a variety of agents and, indirectly, events resulting from such effects.

GML, or a related compound, can inhibit infection of a vertebrate subject by inhibiting infection of a cell of the subject by a relevant infectious microorganism (most likely by inhibiting entry of the infectious microorganism into the cell). Alternatively, GML, or a related compound, can inhibit infection of a vertebrate subject by inhibiting the ability of an infectious microorganism in the subject, or by a microbial factor associated with such an infectious microorganism, to induce the production by a cell of the subject of one or more inflammatory or immunostimulatory mediators that can enhance infectious processes in vertebrate subjects. GML, and related compounds, can act by, for example, binding to the cell membrane of a vertebrate cell and inhibiting any of a variety of vertebrate cellular processes that result in the production by the cell of one or more inflammatory or immunostimulatory mediators after binding of an infectious microorganism or a microbial factor to the cell membrane of the vertebrate cell. Such cellular processes include, for example, cell activation, cell lysis, apoptosis, or cell necrosis. Inflammatory and immunostimulatory mediators include, without limitation, cytokines, chemokines, growth factors, or any of multiple cell components released upon activation, necrosis, lysis, or apoptosis of a vertebrate cell that, by any of a variety of mechanisms, serve to enhance infection of the subject by the relevant microorganism. Such mechanisms include, without limitation, enhancement of extracellular replication of microorganisms, enhancement of cellular uptake of microorganisms and/or of intracellular replication of microorganisms, enhanced translocation of microorganisms between body compartments, enhanced blood flow to a site of infection, enhanced proliferation of host cells (e.g., host cells infected with a virus), enhanced production of other inflammatory and/or immunostimulatory mediators, and/or a localized or systemic increase in body temperature. Inflammatory and immunostimulatory mediators of interest include, without limitation, tumor necrosis factor-α [TNF-α], interleukin [IL]-1β, interferon-γ [IFN-γ], macrophage inflammatory protein-3α [MIP-3α], IL-6, and IL-8.

In addition, or alternatively, GML (or related compounds) can act indirectly to inhibit infection of a vertebrate subject by inhibiting the ability of a cell of the subject (i.e., in a host cell) to respond to an endogenous inflammatory or immunostimulatory mediator (e.g., any of those listed above) produced by the host cell, or another cell of the subject, in response to an infectious microorganism, or a microbial factor. By inhibiting responsiveness of the host cell to such a mediator, GML (or a related compound) can inhibit infection of the subject by: (i) inhibiting infection of the host cell; or (ii) inhibiting production by the host cell of other inflammatory and/or immunostimulatory mediators.

Thus, GML (or related compounds) can be effective therapeutic and/or prophylactic agents against infection by a variety of infectious microorganisms. Moreover, in view of its ability to inhibit cell membrane-mediated effects (e.g., receptor mediated or non-specific cell membrane-binding effects), GML (and related compounds) can be effective against undesirable vertebrate cell death, toxicity to vertebrate cells, inflammatory or immune responses due to microbial agents or non-microbial factors (e.g., plant or animal molecules), even in cases where the activating entity per se is unidentified. Hence GML (and related compounds) can be used to treat a variety of conditions involving undesirable inflammatory and/or immune responses, whether these responses are associated with an infectious microorganism or some other microbial or non-microbial inflammatory or antigenic agent.

As used herein, an agent (e.g., an infectious microorganism, a microbial factor, a non-microbial factor, or a vertebrate host factor) that has a “cell membrane-mediated effect” on a vertebrate cell and is associated with a pathologic condition in a vertebrate subject is an agent that, by a direct or indirect mechanism involving the cell membrane of the vertebrate cell (see above), causes (i) activation, death, lysis, or apoptosis of a vertebrate cell; (ii) facilitation of entry of an infectious microorganism, a microbial factor, a non-microbial factor, or a vertebrate mediator, into a vertebrate cell; (iii) enhanced ability of cell membrane components (e.g., receptors) to bind to infectious microorganisms, microbial factors, non-microbial factors, or vertebrate host mediators; and/or (iv) enhanced permeability of vertebrate cell membranes to infectious microorganisms, microbial factors, non-microbial factors, or vertebrate host mediators.

Relevant pathologic conditions can be due directly to the effect of any of the above-listed agents on a vertebrate cell to whose cell membrane the agent has bound, e.g., immunodeficiency diseases, such as AIDS, resulting from T and/or B cell necrosis or apoptosis induced by an agent, such as HIV-1 or 2, physically interacting with the cell membranes of the T and/or B cells. Alternatively, the pathologic conditions can be indirectly due to such a physical interaction and result from mediators produced by vertebrate cells as a result of the interaction, e.g., mediators released by a vertebrate cell (e.g., macrophages, monocytes and/or epithelial cells) to whose cell membrane the agent has bound.

“Activation of a vertebrate cell” includes causing the cell to grow, enhancing growth of the cell, causing the cell to proliferate (i.e., divide), enhancing proliferation (i.e., division of the cell), causing the cell to produce soluble mediators (e.g., inflammatory or immunostimulatory mediators), and/or enhancing production of such soluble mediators by the cell.

Various aspects of the invention are discussed below.

Methods of Treating Cells with Glycerol Monolaurate (GML) and Related Compounds

The methods of the invention include contacting vertebrate cells with GML, and/or related compounds, so as to inhibit cell membrane-mediated effects (e.g., see above) on the cells by any of a wide range of infectious microorganisms or molecules of microbial or non-microbial origin. As used herein, “infection of a vertebrate cell by an infectious microorganism” means entry of the infectious microorganism into the cell followed, optionally, by replication of the infectious microorganism in the vertebrate cell. “Inhibition of infection of the vertebrate cell” means inhibition of entry of the infectious microorganism into, and/or replication of the infectious microoganism in, the vertebrate cell.

Vertebrate cells that can be treated with GML, or a related compound, can be any vertebrate cell in which GML (or a related compound) can inhibit a cell membrane-mediated effect of an infectious microorganism, a microbial factor, or non-microbial factor (e.g., allergen or host cell mediator). Such cells include epithelial cells (e.g., skin epithelial cells such as keratinocytes; epithelial cells on mucosal surfaces such as nasal, buccal, tracheal, bronchial, pulmonary, anal, rectal, vaginal, or urethral surfaces; bladder epithelial cells; uterine epithelial cells; gastrointestinal (e.g., stomach or colon) epithelial cells; kidney epithelial cells; liver epithelial cells; neural (e.g., brain) epithelial cells; or breast epithelial cells); RBCs, and lymphocytes, e.g., T (CD4+ and CD8+) cells and B cells. In view of the broad activity of GML observed by the inventors (i.e., its action on a wide range of vertebrate cells such as epithelial cells, lymphocytes, and RBCs), it is expected that GML, and related products, will act as described herein on additional cell types, e.g., monocytes/macrophages, any muscle cell, and sperm. Vertebrate (e.g., mammalian) cells can be from any vertebrate species, e.g., humans, non-human primates (e.g., monkeys, chimpanzees, and baboons), horses, cattle, pigs, sheep, goats, dogs, cats, rabbits, guinea pigs, hamsters, gerbils, rats, mice, and birds such as chickens, turkeys, and canaries.

With respect to epithelial cells, appropriate infectious microorganisms include any infectious microorganism that can infect and/or activate any of the epithelial cells listed herein. Bacteria of interest include, without limitation, Staphylococci (e.g., S. aureus, S. intermedius, S. epidermidis, and other coagulase negative Staphylococci), Neisseriae (e.g., N. gonorrheae and N. meningitidis), Streptococci (e.g., Group A Streptococcus (e.g., S. pyogenes), Group B Streptococcus (e.g., S. agalactiae), Group C Streptococcus, Group G Streptococcus, S. pneumoniae, and viridans Streptococci), Chlamydia trachomatis, Treponemae (e.g., T. pallidum, T. pertenue, and T. cerateum), Haemophilus bacteria (e.g., H. ducreyi, H. influenzae, and H. aegyptius), Bordetellae (e.g., B. pertussis, B. parapertussis, and B. bronchiseptica), Gardnerella vaginalis, Bacillus (e.g., B. anthracis and B. cereus), and Clostridium (e.g. C. perfringens, C. septicum, C. novyi, and C. tetani), Escherichia coli, Vibrio cholerae, Salmonella bacteria (e.g., S. enteriditis, S. typhimurium, and S. typhi), Shigella bacteria, Mycobacteria, Francisella bacteria, Yersinia bacteria (e.g. Y. pestis), Burkholderia bacteria, Pseudomonas bacteria, and Brucella bacteria. Mycoplasmal organisms of interest include M. pneumoniae, M. fernentans, M. hominis, and M. penetrans. Fungal organisms (including yeasts) of interest include, without limitation, Candida albicans, other Candida species, Cryptococcus neofomans, and Pneumocyctis carinii. Protozoans of interest include, without limitation, Trichomonas vaginalis. Relevant viruses include, without limitation, rhinoviruses, influenza virus, parainfluenza virus, respiratory syncytial virus, adenoviruses, parvoviruses (e.g., parvovirus B19), roseola virus, enteroviruses, papilloma viruses, retroviruses, herpesviruses (e.g., herpes simplex virus, varicella zoster virus, Epstein Barr virus (EBV), human cytomegalovirus (CMV), and human herpesvirus 6, 7 and 8), and poxviruses (e.g., variola major and variola minor, vaccinia, and monkeypox virus).

Factors that can cause cell membrane-mediated effects on epithelial cells so as to cause or enhance pathological conditions that can be ameliorated or ablated by GML (and related compounds) include microbial and non-microbial factors. Microbial factors include molecules produced or contained by the above-listed bacteria (e.g., exotoxins, endotoxins, or other factors) and viruses that infect and/or activate epithelial cells. Such factors include, but are not limited to, proteases, nucleases, peptidoglycan, lipoteichoic and teichoic acids, protein A, molecules in capsule and slime layers, and bacterial cell adhesion molecules; related factors from other bacteria; LPS from gram negative bacteria; molecules in pili; outer membrane proteins; and molecules in flagella from producing organisms. Other microbial factors of interest are superantigens, allergens, or exotoxins, and can include, without limitation, TSST-1, Staphylococcal alpha, beta, gamma, and delta hemolysins, Streptococcal pyrogenic exotoxins (SPEs), Staphylococcal enterotoxins (SEs; such as SEA, SEB, SEC, or SEE), A-B toxins, Diptheria exotoxin, Cholera exotoxin, Pertussis exotoxin, Shiga toxin, Shiga-like toxin, anthrax (B. anthracis) toxin, Botulinal exotoxin, Tetanus exotoxin, tracheal cytotoxin, Helicobacter toxins, alpha toxin (lecithinase), kappa toxin (collagenase), mu toxin (hyaluronidase), leukocidin, elastase, and fungal toxins. Non-microbial factors of interest include, but are not limited to, nickel and other metal complexes, latex, and molecules in wool, poison ivy, poison oak, poison sumac, plant toxins such as ricin, and animal (e.g., insects such spiders and reptiles such as snakes) venoms. Conditions that are caused by contact of epithelial cells with such factors (e.g., microbial or non-microbial) include, for example, contact dermatitis (e.g., contact dermatitis caused by poison ivy) the papules and pustules that arise in rosacea [Dahl et al. (2004) J. Am. Acad. Dermatol. 50:266-272], toxic shock syndrome (TSS), pneumonia, bacteremia in association with cutaneous infection (e.g., cellulitis, erysipelas, or infection of a surgical or nonsurgical wound), deep soft tissue infection (e.g., myositis or necrotizing fasciitis), meningitis, peritonitis, osteomyelitis, septic arthritis, postpartum sepsis (e.g., puerperal fever), or neonatal sepsis.

With respect to T cells, appropriate infectious microorganisms include any infectious microorganisms that cause a cell membrane-mediated effect on CD4+ and/or CD8+ T cells. Bacteria of interest include any of those listed above for epithelial cells. Relevant viruses, in addition to those listed above for epithelial cells, include retroviruses such as human immunodeficiency virus (HIV)-1 and 2, human lymphotropic virus (HTLV) 1 and 2, feline leukemia virus, superantigen gene-containing viruses such as murine mammary tumor viruses, and herpesviruses such as human herpesviruses 6 and 7 as well as EBV which has been shown to activate expression of an endogenous superantigen in humans [Sutkowski et al. (2001) Immunity 15(4):579-589].

Factors that activate T cells so as to cause cell membrane-mediated effects, which can be inhibited by GML and related compounds, include microbial and non-microbial factors. Microbial factors include molecules produced by, or contained within, or on the surface of the above-listed bacteria (e.g., exotoxins, endotoxins, or other factors) and viruses that infect and/or activate T cells. Microbial factors include the superantigens (e.g., the Staphylococcal enterotoxins, TSST-1, Streptococcal pyrogenic exotoxins, and type 5 M protein) as well as non-superantigen molecules produced by the above-listed bacteria and viruses that activate and/or infect T cells. Also of interest are any antigens that activate T cells and cause delayed-type hypersensitivity (DTH) reactions. Such antigens, which can be of microbial or non-microbial origin (e.g., food and drugs antigens as well as metallic and plant- and animal-derived substances), activate antigen-specific T cells with symptoms of the response being maximal 24-72 hours after exposure of the subject to the antigen. Examples of relevant antigens include, without limitation, streptokinase, streptococcal DNases, Candida antigens, ring worm fungi antigens, nickel and other metals, and latex, as well as antigens from wool, poison ivy, poison oak, and poison sumac. While CD4+ T cells appear to be the principle mediators of DTH responses, CD8+ T cells also contribute to and modulate such responses. Other non-microbial molecules of relevance to activation of T cells are non-microbial polyclonal T cell activators such as the plant lectins phytohemagglutinin (PHA) and concanavalin A (ConA).

Examples of pathological conditions caused by cell membrane or associated effects on T cells include psoriasis, which is associated with infection by S. aureus and Group A Streptococcus, and atopic dermatitis, which is associated with infection by S. aureus and other gram positive bacteria. In addition, the papules and pustules that occur in rosacea patients have been shown to be associated with S. epidermidis and acne is caused by Propionibacterium acnes.

With respect to B cells, appropriate infectious microorganisms include any infectious microorganism that can infect and/or activate B cells. Included in this category would be gram negative bacteria that produce lipopolysaccharide (LPS), an endotoxic molecule that activates B cells. Examples of such bacteria include, without limitation, Salmonella, Shigella, Escherichia (e.g., E. coli), Enterobacteriaceae, Vibrio (e.g., V. cholerae), Pseudomonas, Neisseria (see above), and Haemophilus (see above). Viruses of interest include herpesviruses such as EBV and herpesvirus 8.

Factors that cause cell membrane-mediated effects on B cells so as to cause undesirable pathological conditions (e.g., enhanced inflammatory or immune responses) that can be inhibited by GML and related compounds include microbial and non-microbial factors. Microbial factors include LPS as well as other molecules produced by the above-listed bacteria and viruses that infect and/or activate B cells. Also of interest are any of the antigens that activate antigen-specific B cells to produce antibodies (e.g., IgE antibodies) and that cause immediate-type hypersensitivity reactions on re-exposure of the subject to the antigens. These antigens can be of microbial or non-microbial origin (e.g., certain drugs (e.g., penicillin) as well as plant- and animal-derived substances such as molecules in ragweed pollen and other pollens or cat dander). Non-microbial factors include any of the molecules listed for T cells, epithelial cells, monocyte/macrophages, and RBCs. Immediate hypersensitivity response symptoms are generally maximal from a few minutes up to 24 hours after exposure of the subject to the antigen. Other non-microbial molecules of relevance to B cells are non-microbial polyclonal B cell activators such as the plant lectin, pokeweed mitogen (PWM).

With respect to monocytes/macrophages, appropriate infectious microorganisms include any infectious microorganism that can infect and/or activate monocytes/macrophages. Bacteria in this category include gram negative bacteria that produce lipopolysaccharide (LPS) (see above) that activates monocytes/macrophages, staphylococci and streptococci, Listeria monocytogenes. Viruses of interest include HIV and any of those listed above.

Factors that cause cell membrane-mediated effects on monocytes/macrophages that are associated with undesirable pathological conditions (e.g., enhanced inflammatory or immune responses) that can be inhibited by GML (and related compounds) include microbial and non-microbial factors. Such factors include LPS, which is the cause of septic shock in subjects with severe gram negative bacterial infections, as well as other molecules produced by the above-listed bacteria and viruses that infect and/or activate monocytes/macrophages, e.g., flagella molecules, peptidoglycan, superantigens, and double stranded DNA and RNA.

With respect to RBCs, appropriate infectious microorganisms include any infectious microorganism that can infect an RBC. Appropriate microorganisms include, for example, protozoans that have erythrocytic stages, e.g., malarial protozoans such as Plasmodium falciparum, P. vivax, P. ovale, and P. malariae.

With respect to RBCs, factors that cause cell membrane-mediated effects on RBCs that are associated with undesirable pathological conditions that can be inhibited by GML (and related compounds) include microbial and non-microbial factors. Microbial factors include any of the infectious microorganisms listed and can be parasites, for example, malaria. Microbial factors can also include factors that are produced or components of infectious microorganisms. Microbial factors can be exotoxins, and include, without limitation, TSST-1, Staphylococcal alpha, beta, gamma, and delta hemolysins, Streptococcal pyrogenic exotoxins (SPEs), Staphylococcal enterotoxin B (SEB), A-B toxins, Diptheria exotoxin, Cholera exotoxin, Pertussis exotoxin, Shiga toxin, Shiga-like toxin, antrax toxin, Botulinal exotoxin, Tetanus exotoxin, tracheal cytotoxin, Helicobacter toxins, alpha toxin (lecithinase), kappa toxin (collagenase), mu toxin (hyaluronidase), leukocidin, and elastase. Non-microbial factors include any molecules or factors of non-biological origin that can cause or enhance a cell membrane-mediated effect of a cell of a vertebrate subject. Non-microbial factors include any of those listed above. Pathological conditions caused by cell membrane or associated effects on RBCs by microbial factors include, without limitation, anemia of chronic disease (ACD), megaloblastic anemia, or other RBCs disorders or dysfunctions that are caused by any of the above-described factors.

Other factors whose pathology-related cell membrane-mediated effects can be inhibited by GML (and related compounds) include endogenous vertebrate factors that can exert their effects against any appropriate vertebrate target cell (including any of those recited herein) such as: (a) complement which is a series of factors that ultimately cause lysis of a target cell by binding to and forming pores in the cell membranes of cells, for example, to which an antibody (especially an IgM antibody) has bound; and (b) perforins and natural killer cytotoxic factor (NKCF) which are effector molecules of cytotoxic T lymphocytes (CTL) and natural killer (NK) cells and that act to kill appropriate target cells by forming pores in the cell membranes of the target cells. Complement-mediated cell membrane-mediated effects are relevant to pathologic conditions involving host cell-specific antibodies including autoimmune diseases (e.g., systemic lupus erythematosus), hyperacute rejection of transplanted organs and tissues (especially xenogeneic transplantation), and complement mediated hypersensitivity reactions. CTL are involved in pathologic conditions such as, for example, chronic rejection of both allogeneic and xenogeneic grafts and autoimmune diseases such as insulin-dependent diabetes mellitus (IDDM), multiple sclerosis (MS), and rheumatoid arthritis (RA). NK cells have been implicated in the rejection of stem cell (e.g., bone marrow) transplants.

In addition, in view of the broad cell-type activity of GML, it is likely that it, and related compounds, inhibit (or even prevent) cell membrane-mediated effects of sperm that are required for entry of sperm into ova and thereby prevent fertilization of ova. In light of such activity, GML and related compounds can be useful as spermicides.

The compounds that can be used to inhibit the above-described infection and/or activation of cells include a variety of glycerol-based compounds related to GML. The compounds include fatty acid esters of glycerol in which the alcohol group on one, or both, of the terminal carbon atoms of glycerol, the alcohol group on only the middle carbon atom, the alcohol groups on the middle carbon atom and one of the terminal carbon atoms, or the alcohol groups on all three carbon atoms are esterified with fatty acids. The fatty acids can be 10 carbon, 11 carbon, 12 carbon, 13 carbon, or 14 carbon linear alkyl fatty acid esters and can be present in the molecule in any combination. In addition, instead of being linked to the glycerol backbone by ester linkages, 10 carbon, 11 carbon, 12 carbon, 13 carbon, or 14 carbon linear alkyl chains can be linked to it by ether linkages.

In summary, the inhibitory glycerol-based compounds useful in the invention include the following: 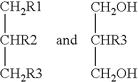
in which R1 is: OH; CO(CH₂)₈CH₃; CO(CH₂)₉CH₃; CO(CH₂)₁₀CH₃; CO(CH₂)₁₁CH₃: CO(CH₂)₁₂CH₃; O(CH₂)₉CH₃; O(CH₂)₁₀CH₃;O(CH₂)₁₁CH₃; O(CH₂)₁₂CH₃; or O(CH₂)₁₃CH₃,
R2 is: OH; CO(CH₂)₈CH₃; CO(CH₂)₉CH₃; CO(CH₂)₁₀CH₃; CO(CH₂)₁₁CH₃: CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃, and
R3 is: CO(CH₂)₈CH₃; CO(CH₂)₉CH₃; CO(CH₁₂)₁₀CH₃; CO(CH₂)₁₁CH₃: CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃.

Additional inhibitor compounds useful in the invention include, for example: (a) phosphatidyl choline and phosphatidyl ethanolamine, and (b) sphingolipids such as ceramides. In these compounds, the fatty acids (or corresponding ether-linked linear alkyl chains) are any of those described above.

One or more (e.g., two, three, four, five, six, seven, eight, nine, ten, 11, 12, 15, 18, 20, 25, 30, or more) of the above compounds can be delivered to a vertebrate cell of interest, either alone, or with one or more (e.g., two, three, four, five, six, seven, eight, nine, ten, 11, 12, 15, 18, 20, 25, 30, or more) supplementary agents. Such supplementary agents include substances that serve, for example, to inhibit cell membrane or cell membrane-associated effects. For example, supplementary factors may inhibit infection (e.g., standard anti-microbial antibiotics) or inhibit activation of any of the above-described vertebrate cell populations, e.g., immunoregulatory cytokines or antibodies specific for such cytokines. For example, where it is desired to decrease a Th1-type immune response (e.g., in a DTH response), a cytokine such as interleukin (IL)-4, IL-10, or IL-13 or an antibody specific for a cytokine such as IL-12 or interferon-γ (IFN-γ) can be used as a supplementary agent. Alternatively, where it is desired to inhibit a Th2-type immune response (e.g., in an immediate type hypersensitivity response), a cytokine such as IL-12 or IFN-γ or an antibody specific for IL-4, IL-10, or IL-13 can be used as a supplementary agent. Also of interest are antibodies specific for proinflammatory cytokines and chemokines such as IL-1, IL-6, IL-8, TNF-α, MIP-1, MIP-3α, monocyte chemoattractant protein-1 (MCP-1), epithelial neutrophil activating peptide-78 (ENA-78), interferon-γ inducible protein-10 (IP10), Rantes, and any other appropriate cytokine or chemokine recited herein.

All the antibodies described above can be polyclonal antibodies or monoclonal antibodies (mAb) and can be from any of a wide range of species, e.g., a human, a non-human primate (e.g., a monkey or a chimpanzee), a cow, a horse, a goat, a sheep, a pig, a cat, a dog, a rabbit, a guinea pig, a hamster, a gerbil, a rat, a mouse, or a chicken.

As used herein, the term “antibody” refers to any class of antibody (e.g., IgM, IgG, IgA, IgD, or IgE). Also useful for the invention are antigen-binding fragments, e.g., Fab, F(ab′)₂, Fv, and single chain Fv (scFv) fragments. An scFv fragment is a single polypeptide chain that includes both the heavy and light chain variable regions of the antibody from which the scFv is derived. Also included are chimeric antibodies, e.g., humanized antibodies.

Antibody fragments that contain the binding domain of the molecule can be generated by known techniques. For example: F(ab′)₂ fragments can be produced by pepsin digestion of antibody molecules; and Fab fragments can be generated by reducing the disulfide bridges of F(ab′)₂ fragments or by treating antibody molecules with papain and a reducing agent. See, e.g., National Institutes of Health, 1 Current Protocols In Immunology, Coligan et al., ed. 2.8, 2.10 (Wiley Interscience, 1991). scFv fragments can be produced, for example, as described in U.S. Pat. No. 4,642,334, which is incorporated herein by reference in its entirety.

Chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 173,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1987) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60.

Fully human antibodies (polyclonal or monoclonal) can be produced by immunizing transgenic animals (e.g., mice) that contain gene segments encoding gene segments encoding all human immunoglobulin (i.e., variable, joining, diversity, and constant) regions (see, for example, U.S. Pat. Nos. 5,545,806 and 5,569,825).

The methods of inhibiting infection and/or activation of a cell can be in vitro or in vivo.

In vitro application of the methods of the invention can be useful in basic scientific studies of infection, mechanisms of cellular resistance to infection, inflammation, and methods of controlling infection and/or inflammation. In the in vitro methods of the invention, one or more inhibitory compounds can be cultured with vertebrate cells of interest (see above) and any of the infectious microorganisms, or factors produced by produced by such infectious microorganisms, described above. Such cultures can also be “positive controls” in screening assays for new inhibitory compounds. Moreover, in vitro method can be those in which it is desired to culture relevant vertebrate cells (e.g., epithelial cells) with an infectious microorganism of interest but without, or minimizing, infection the vertebrate cells. Measurements of, for example, infectious microorganism titer, level of cell proliferation/survival, can be made after various times of incubation using methods known in the art. These in vitro systems can contain, in addition to inhibitory compounds, one or more of the supplementary agents described above.

The methods of the invention will preferably be in vivo. These applications can be useful in the therapy and prophylaxis of infectious diseases that are associated with cell membrane-mediated effects in vertebrate cells caused by any of the infectious microorganisms, or microbial products, recited herein. They can also be useful for diminishing the side effects of vaccination with, for example, a live virus (e.g., cowpox). By administering a compound of interest to an animal infected (e.g., a human patient with an S. aureus infection), an animal that will be infected (e.g., a human subject to be vaccinated with vaccinia virus), or an animal at risk of being infected (e.g., a human subject at risk of being infected by a B. anthracis in the course of an anticipated bioterrorist attack), therapy of, or prophylaxis from, the clinical symptoms caused by the virus can be achieved.

As used herein, “prophylaxis” can mean complete prevention of the symptoms of a disease, a delay in onset of the symptoms of a disease, or a lessening in the severity of subsequently developed disease symptoms. As used herein, “therapy” can mean a complete abolishment of the symptoms of a disease or a decrease in the severity of the symptoms of the disease.

The methods of the invention can be applied to a wide range of species, e.g., humans, non-human primates (e.g., monkeys, chimpanzees, and baboons), horses, cattle, pigs, sheep, goats, dogs, cats, rabbits, guinea pigs, hamsters, gerbils, rats, mice, and birds such as chickens, turkeys and canaries.

In Vivo Approaches

In a preferred in vivo approach, one or more of the isolated compounds is administered to the subject. In addition, one or more of the above-described supplementary agents can be administered together with, or separate from, the inhibitory compounds. Where the supplementary agents are administered separately, they can be administered simultaneously with the compounds but by a different route. Alternatively, they can be administered at a different time from the inhibitory compounds and either by the same route or by a different route.

Generally, the compounds and supplementary agents will be suspended in a pharmaceutically-acceptable carrier (e.g., physiological saline) and administered orally or by intravenous (i.v.) infusion, or injected subcutaneously, intramuscularly, intrathecally, intraperitoneally, intrarectally (e.g., in suppositories), intravaginally, intranasally, intragastrically, intratracheally, or intrapulmonarily. They can, for example, be delivered directly to a site of infection and/or inflammation, e.g., intrapulmonarily where the infection and/or inflammation is of the lung. Moreover, the compounds can be administered topically. For topical application, the compounds can be incorporated into, for example, creams, gels, creams, foams, cosmetics, shampoos, toothpastes, or bath soaps. They can be used in, for example, acne medications or in spermicidal compositions, e.g., gels, foams, or creams. The dosages of the inhibitory compounds and supplementary agents to be used depend on the choice of the route of administration; the nature of the formulation; the nature of the patient's illness; the subject's size, weight, surface area, age, and sex; other drugs being administered; and the judgment of the attending physician. Suitable dosages are in the range of 0.0001-100.0 mg/kg. Wide variations in the needed dosage are to be expected in view of the variety of compounds and supplementary available and the differing efficiencies of various routes of administration. For example, oral administration would be expected to require higher dosages than administration by i.v. injection. Variations in these dosage levels can be adjusted using standard empirical routines for optimization as is well understood in the art. Administrations of compounds and/or supplementary agents can be single or multiple (e.g., 2-, 3-, 4-, 6-, 8-, 10-, 20-, 50-,100-, 150-, or more fold). Encapsulation of the compounds and/or supplementary agents in suitable delivery vehicles (e.g., polymeric microparticles, implantable devices, or suppositories) may increase the efficiency of delivery.

Methods to test whether a particular regimen is therapeutic for, or prophylactic against, a particular pathologic condition are known in the art. Where a therapeutic effect is being tested, a test population displaying symptoms of the pathologic condition (e.g., humans or experimental animals having a S. aureus infection) is treated with a test regimen involving of any of the above-described strategies. A control population, also displaying symptoms of the pathologic condition, is treated, with a placebo or a different regimen. Disappearance or a decrease of symptoms of the pathologic condition in the test subjects would indicate that the test regimen is an effective therapeutic methodology.

By applying the same strategies to subjects prior to onset of symptoms of the pathologic condition (e.g., experimental animals prior to deliberate infection with a herpesvirus), test regimens can be tested for efficacy as prophylactic methodologies. In this situation, prevention of, or delay in, onset of symptoms of the pathologic condition is tested.

The following examples are meant illustrate, not limit, the invention. O 2005/047475 PCT/US2004/037466

### EXAMPLES

### Example 1

### Materials and Methods

### Bacteria

S. aureus strain MN8 is a typical menstrual TSS isolate that is positive for (i.e., expresses) TSS (toxic shock syndrome) toxin-1 (TSST-1). Low passage samples of the organism are maintained in the inventors' laboratory in a lyophilized state. It was determined experimentally that S. aureus at a cell concentration 1×10⁹/ml corresponded to an absorbance at 600 nm of 1.2.

The N. gonorrhoeae strain used in the studies described herein was a clinical isolate from acute gonorrhea. The organism was stored frozen at −80° C. in the presence of 10% glycerin. N. gonorrhoeae a cell concentration of 2×10⁹ μl corresponded to an absorbance at 600 nm of 1.0.

C. trachomatis serotype e elementary bodies (the infectious form of the bacterium) were obtained from Dr. Gerald Byrne of the University of Tennessee, Memphis, Tenn., and were stored at −80° C. in the presence of 10% glycerin.

The Group A Streptococcal strain T18P (M type 18) used in the experiments described below was a Group A Streptococcal isolate associated with an outbreak of pharyngitis and rheumatic fever. Low passage samples of the organism are maintained in the inventors' laboratory in a lyophilized state. It was determined experimentally that Group A Streptococcus T18P at 5×10⁸ CFU (colony-forming units)/ml corresponded to an absorbance at 600 nm of 1.5.

The day prior to use, S. aureus MN8 and Group A Streptococcus T18P organisms were subcultured onto blood agar plates and N. gonorrhoeae onto chocolate agar plates. Chlamydia elementary bodies were used directly as supplied. For epithelial infection experiments, bacterial cells (except Chlamydia) were used at initial concentrations of 100 and 1000 bacteria/epithelial cell. The bacteria were prepared for use by scraping them directly from blood or chocolate agar plates into 10 ml of keratinocyte serum-free medium KSFM; Gibco Life Technologies, Carlsbad, Calif.) without antibiotics, washing them one time with 10 ml of KSFM without antibiotics, and adjusting them to the indicated concentration based on absorbance at 600 nm; actual cell counts added to epithelial cells were determined subsequently by plating, culturing, and colony counting. The amount of GML required to inhibit the growth of S. aureus and N. gonorrhoeae was determined by incorporation of the compound into either Todd Hewitt (for S. aureus) or chocolate (for N. gonorrhoeae) plates and then incubating the plates for 48 h with the organisms streaked onto the surface.

For microarray experiments, S. aureus MN8 was used at a cell density of 1×10⁹ bacteria/3×10⁷ epithelial cells in total volumes of 10 ml.

### Glycerol Monolaurate (GML)

GML pellets (manufactured as Monomuls 90 L-12 by Cognis Henkel Eco-Labs, Germany) were dissolved in absolute ethanol at a concentration of 100 mg/ml. The compound was diluted in ethanol from this stock solution for use such that the maximum volume/volume addition to cultures was 10 μl GML solution per ml medium. The same volume of ethanol without GML was added to control cultures.

### Generation of Immortal Human Vaginal Epithelial Cells

Immortal human vaginal epithelial cells were a gift from Dr. Kevin Ault of University of Iowa, Iowa City, Iowa, and were generated as described below.

Primary normal human epithelial cells were isolated from premenopausal vaginal hysterectomy tissue obtained from a patient who did not have cancer using methods that have been previously described for the isolation of human foreskin epithelial cells [Halbert et al. (1992) J. Virol. 66:2125-2134]. The E6 and E7 genes of human papillomavirus type 6 have weak immortalizing activity in human epithelial cells. Cells were grown in KSFM (Gibco Life Technologies) on plastic and passaged with a 1:4 split using trypsin-EDTA solution (1× trypsin-EDTA (ethylene diamine tetraacetic acid); 0.25% trypsin, 0.1% EDTA; Mediatech, Inc., Herndon, Va.). Early passage cells were doubly transduced with retroviruses expressing HPV-16 E6/E7 (a gift from Dr. Denise Galloway, Fred Hutchinson Cancer Research Center, Seattle, WN) and the reverse transcriptase component of telomerase, hTERT (obtained from the Geron Corporation), and selected in 50 μg/ml G418 as previously described [Kiyono et al. (1998) Nature 396:84-88]. Both Rb/p16^{INK4a} inactivation and telomerase activity are required to immortalize human epithelial cells [Kiyono et al. (1998)]. Cells surviving selection (V428) had high levels of telomerase and became immortal without crisis, whereas normal untransduced cells senesced at about passage 9.

### Epithelial Cell Infection Experiments

V428 epithelial cells were grown to confluence on the well bottoms of 6 well, flat-bottomed microtiter plates (experimentally determined to be 9.6×10⁵ cells/well at confluence) in the presence of KSFM. The day before use, the KSFM was removed, and 2 ml of fresh KSFM were added to each well. On the day of use, the KSFM was removed, and 1 ml of fresh KSFM containing GML dissolved in ethanol or ethanol alone was added to each well. After a 1-hour incubation at 37° C. in an atmosphere of 7% CO₂, bacteria (in 10 μl volumes) were added to the tissue culture wells which were then incubated for 3 hours at 37° C., 7% CO₂. The wells were then washed 2 times with KSFM, incubated with 2 ml KSFM containing 20 μg/ml gentamicin for 1 hour to kill residual extracellular bacteria, and then washed three additional times with 2 ml KSFM to remove gentamicin. The epithelial cells were then removed from the plates by scraping with Falcon single use cell scrapers (BD Biosciences, Bedford, Mass.). Bacterial colony forming units (CFU) were determined by plate counts on combinations of blood and chocolate agars for S. aureus and chocolate agar for N. gonorrhoeae. The experiment with Group A Streptococcus T18P was performed using an essentially identical protocol.

An experiment to test for the effect of GML on infection by C. trachomatis was performed essentially as described above, except that the plates were incubated for 24 hours (rather than 3 hours) after addition of the bacteria and, after was washing the plates to remove gentamycin, the epithelial cells were fixed with ethanol and then Giemsa stained. Inclusion bodies were counted by light microscopy.

### Microarray Experiments

V428 human vaginal epithelial cells were grown to confluence in 250 ml Falcon tissue culture flasks (BD Biosciences) (approximately 3×10⁷ cells/flask) in KSFM. The medium was removed and replaced with new medium the day before experimentation. On the day of assay, S. aureus MN8 was added to epithelial cells at a bacterial concentration of 10⁹/ml KSFM added, i.e., 10¹⁰ bacteria in a total volume of 10 ml KSFM. Separate flasks were then incubated for 3 and 6 hours in the absence and presence of GML (1000 μg/ml). All incubations were stationary, at 37° C., and in an atmosphere of 7% CO₂. At the end of the incubation, flasks were removed from the incubator, 10 ml trypsin-EDTA solution (1× trypsin-EDTA; 0.25% trypsin, 0.1% EDTA; Mediatech, Inc.) was added to the flasks, and after 5 minutes detached epithelial cells were removed from the flasks. The epithelial cells were washed once and RNA was isolated from them using the Qiagen RNeasy Mini™ kit. Fragmented biotin-labeled cRNA was prepared from this RNA used for microarray analysis using methods described in the GeneChip® Expression Analysis Technical Manual (Affymetrix, Santa Clara, Calif.; April, 2003). In brief, single-stranded cDNA was prepared from the RNA, double-stranded cDNA was prepared from the single-stranded cDNA, and biotin-labeled cRNA was prepared from the double-stranded cDNA. Finally, the biotin-labeled cRNA was fragmented by metal-induced hydrolysis and allowed to hybridize to an Affymetrix U133A Human GeneChip® (Affymetrix). Data were analyzed by software provided by The Institute for Genome Research (Microarray Software Suite; Rockville, Md.) and Affymetrix Microarray Suite Software.

### Lymphocyte Proliferation Experiments

Human peripheral blood mononuclear cells (PBMC) were isolated from human blood by density gradient centrifugation. PBMC adhering to plastic tissue culture Petri dishes were used as antigen-presenting cells (APC). PBMC that did not adhere to the tissue culture dishes were applied to a Sephadex™ G-10 column and T cells were purified from cells passing through the column by two sequential rounds of sheep red cell rosetting (“E-rosetting”), density gradient centrifugation, and lysing of sheep red cells with ammonium chloride (0.15 M). T cells (1×10⁵ per well) and APC (3×10⁴ per well) were plated into the wells of 96-well tissue culture microtiter plates without and with TSST-1 (10 μg/ml) and without and with GML (15 μg/ml). The plates were incubated for 3 days at 37° C., pulsed with ³H-thymidine (1 μCi), and incubated at 37° C. for another 24 hours. The cells were harvested and the relative levels of cell proliferation determined in terms of the amount of ³H-thymidine (counts per minute; cpm) incorporated into the cells. Radioactivity was measured using a scintillation counter (Beckman Instruments, Fullerton, Calif.).

BALB/c mouse spleen cells adhering to plastic tissue culture Petri dishes were used as antigen-presenting cells (APC). B cells were purified from spleen cells that did not adhere to the tissue culture dishes by lysis of red blood cells and killing of T cells with antibodies specific for T cells and complement. B cells (1×10⁵ per well) and APC (3×10⁴ per well) were plated into the wells of 96-well tissue culture microtiter plates without and with lipopolysaccharide (LPS; 10 μg/ml) and without and with GML (15 μg/ml). The plates were incubated for 3 days at 37° C., pulsed with ³H-thymidine (1 μCi), and incubated at 37° C. for another 24 hours. The cells were harvested and the relative levels of cell proliferation determined as described above.

### Example 2

### Effect of GML on Bacterial Growth

Initial studies were undertaken to determine the tolerance of both S. aureus and N. gonorrhoeae to GML. For these studies, media plates containing various amounts of GML were inoculated with bacteria, and the plates were incubated for 48 h and examined for growth. S. aureus MN8 grew on Todd Hewitt plates containing up to 100 μg/ml GML, but some growth was also noted on plates containing 200 μg/ml. The organism did not grow on plates containing 300 μg/ml. N. gonorrhoeae grew on chocolate agar plates containing GML at concentrations up to 50 μg/ml.

### Example 3

### Effect of GML on Bacterial Infection of Epithelial Cells

Both S. aureus and N. gonorrhoeae have the ability to invade and replicate in epithelial cells. It was hypothesized that GML might interfere with invasion of cells by bacteria and in this way block initiation of bacterial infection. Pre-incubation of epithelial cells with either 20 or 50 μg/ml of GML effectively inhibited infection by S. aureus and N. gonorrhoeae (Table 1).

TABLE 1
Inhibition of S. aureus MN8 and N. gonorrhoeae infection
of epithelial cells by GML.

| | | Average^{a} colony forming units | |
|---|---|---|---|
| | | from two wells at bacteria:epithelial cell | |
| | | ratio of: | |
| Bacterium | GML | 100:1 | 1000:1 |
| S. aureus | None | 500 | 525,000 |
| S. aureus | 20 μg/ml | <10^{b} | 350 |
| S. aureus | 50 μg/ml | <10 | <10 |
| N. gonorrhoeae | None | <10 | 3300 |
| N. gonorrhoeae | 20 μg/ml | <10 | <10 |
| N. gonorrhoeae | 50 μg/ml | <10 | <10 |

^{a}An average was taken from two wells per sample. There were approximately 9.6 × 10⁵ epithelial cells/well.
^{b}Lower limit of detection.

S. aureus MN8 at an initial bacterium to epithelial cell ratio of 100:1 invaded and grew to cell densities of 500 bacteria/10⁶ epithelial cells. In contrast, no bacteria were detectable in epithelial cells pre-treated with either GML concentration. When S. aureus MN8 was incubated at a bacterial to epithelial cell ratio of 1 000:1, again significant protection of epithelial cells from S. aureus infection was also seen (Table 1).

Similarly, at a N. gonorrhoeae to epithelial cell ratio of 1000:1, GML, at both concentrations, provided significant protection from infection by N. gonorrhoeae (i.e., no detectable bacteria in GML treated cells compared to 4×10⁴ N. gonorrhoeae/10⁶ epithelial cells for the non-GML treated cells) (Table 1). There were too few N. gonorrhoeae to count in the studies in which a N. gonorrhoeae to epithelial cell ratio of 100:1 was used.

Using essentially the same protocol, an epithelial cell infection experiment was carried out with S. pyogenes. As indicated in Table 2, GML (at concentrations of 10 and 20 ug/ml) resulted in dramatic decreases in the number of bacteria in the epithelial cells.

TABLE 2
Inhibition of Streptococcus pyogenes T18P infection
of epithelial cells by GML.

| | | Average^{a} colony forming units from two | |
|---|---|---|---|
| | | wells at bacteria:epithelial cell ratio of: | |
| Bacterium | GML | 100:1 | 1000:1 |
| S. pyogenes | None | 110 | 42,000 |
| S. pyogenes | 10 μg/ml | <10^{b} | <10 |
| S. pyogenes | 20 μg/ml | <10 | <10 |

^{a}An average was taken from two wells per sample. There were approximately 9.6 × 10⁵ epithelial cells/well.
^{b}Lower limit of detection.

An experiment was performed to test for an effect of GML on infection of the epithelial cells by the obligate intracellular bacterium, C. trachomatis. The GML was added to the epithelial cells in a volume of 1 ml in an amount of 20 μg per well and the bacteria were added to the culture wells at concentrations of 10⁸, 10⁷, 10⁶, and 10⁵ per well (Table 3)

TABLE 3
Inhibition of Chlamydia trachomatis infection
of epithelial cells by GML.

| | | Average number of inclusion | |
|---|---|---|---|
| | Number of bacteria | bodies per field^{a} | |
| Bacterium | added per well | With GML | Without GML |
| C. trachomatis | 10⁸ | None | 54 |
| C. trachomatis | 10⁷ | None | 44 |
| C. trachomatis | 10⁶ | None | 25 |
| C. trachomatis | 10⁵ | None | Occasional |

^{a}An average was determined of the number of inclusion bodies per microscopic field in three separate microscopic fields in each of two culture wells per treatment group. There were approximately 10⁶ epithelial cells per well. Fifty inclusion bodies per field correspond to approximately 10⁴ inclusion bodies per 10⁶ epithelial cells (i.e., per well).

While inclusion bodies were seen in all wells to which no GML was added, no inclusions were seen in wells to which GML was added.

These data suggest that GML inhibits entry of bacteria into epithelial cells. While the experiments in Example 2 suggest that GML (at the concentrations used) did not act by inhibiting intracellular replication of the bacteria, the possibility that it did act in this way cannot be completely excluded. The invention is not limited by any particular mechanism of action. Moreover, the fact that GML inhibited infection by an obligate intracellular pathogen (C. trachomatis) indicates that it is likely that infection by viruses, also obligate intracellular pathogens, is likely also to be inhibited by GML and related compounds.

### Example 4

### A Microarray Analysis of the Epithelial Cell Response to S. aureus Bacteria

In preliminary mRNA microarray analyses it was observed that the V428 vaginal epithelial cells responded to S. aureus by up- or down-regulating the expression of a multiplicity of genes. The expression of 2,889 genes was modulated by 1.5-fold or greater, of 986 genes by 2-fold or greater, and 84 genes by 5-fold or more. Of the 84 genes whose expression was modulated by 5-fold or more, the expression of 83 genes was up-regulated. Of these 83 genes, most are involved in signal transduction and lead to activation of the immune system. For example, the expression of chemokine ligand 20 (macrophage inflammatory protein (MIP)-3α) gene was upregulated by 274-fold, that of interleukin-8 (IL-8) by 64-fold, and that of tumor necrosis factor α (TNFα) by 19.7-fold. Other proinflammatory/immunregulatory genes whose expression was upregulated include chemokine ligand 2 (27.9-fold), chemokine ligand 1 (21.1-fold), the cytokines interleukin (IL)-1α (7.5-fold) and IL-1β (6.1-fold), and the adhesion ligand ICAM-1 (10.6-fold).

The above-described inhibitory effect of GML on bacterial infection of vaginal epithelial cells was likely due to GML altering the epithelial cell membranes so as to prevent entry of the bacteria into the epithelial cells. Thus it seemed very possible that GML could ultimately inhibit, via initial effects on the epithelial cell surface, mRNA synthesis by the epithelial cells. Thus, an experiment was undertaken to assess the effect of GML on RNA synthesis by epithelial cells exposed to 09 S. aureus MN8. Since S. aureus makes a glycerol ester hydrolase, these studies were conducted in the presence of GML at a concentration of 1,000 μg/ml. Although this was a higher concentration of GML than used in the infections studies, the experiment protocols used in the two types of experiments were different. In the gene expression experiment, staphylococcal glycerol ester hydrolase was not removed from the system by the washing out of the bacterial cells, whereas it was removed by washing in the infection studies. Glycerol ester hydrolase produced by S. aureus has been shown to degrade GML.

At times of both 3 and 6 hours, complete inhibition of RNA synthesis in the presence of GML was observed. On the other hand, there was substantial mRNA synthesis in epithelial cells exposed to either S. aureus MN8 alone or epithelial cells incubated in the absence of both GML and S. aureus MN8.

These studies suggest that GML interferes with signal transduction by epithelial cells so as to prevent internalization of S. aureus and N. gonorrhoeae. Moreover, they indicate that GML can inhibit the production of cytokines, chemokines and pro-inflammatory molecules whose expression in epithelial cells is activated by bacteria and in this fashion inhibit the progress of infection by relevant bacteria.

### Example 5

### GML Inhibits the Proliferation of T and B Cells that is Activated by the Bacterial Products TSST-1 and Lipopolysaccharide (LPS)

The data in Table 4 show that GML (at a concentration of 15 μg/ml) inhibits the proliferative response of human T cells to TSST-1 and murine B cells to LPS.

TABLE 4
Inhibitory effect of GML (15 μg/ml) on proliferation
activated by B cell (lipopolysaccharide, LPS) and T cell
(TSST-1) mitogens.

| | Counts/min (incorporation of ³H- |
|---|---|
| Immune Cell Population | thymidine into DNA) |
| T cells plus GML | 5,000 |
| T cells plus GML plus TSST-1 | 5,500 |
| T cells plus TSST-1 | 80,000 |
| B cells plus GML | 5,500 |
| B cells plus GML plus LPS | 10,000 |
| B cells plus LPS | 60,000 |

TSST-1 a potent superantigen, stimulated T cell proliferation. In contrast, when GML was present, TSST-1 did not induce significant T cell proliferation. Similarly, GML inhibited B cell proliferation induced by LPS. These data indicate that GML can inhibit T and B cell-mediated inflammatory responses induced by such microorganisms and/or substances produced by them.

### Example 6

### GML Inhibits Cellular Lysis of Red Blood Cells (RBCs) by Staphylococcal Exotoxin α-Hemolysin

Staphylococcal α-hemolysin is an exotoxin that is toxic to cells and causes their lysis by forming heptamer pores on the cellular membranes of vertebrate target cells. In particular, RBCs are highly sensitive to the toxic effects of α-hemolysin.

The ability of GML to inhibit the lytic effects of staphylococcal α-hemolysin on rabbit RBCs was evaluated. Highly purified α-hemolysin was incubated with rabbit RBCs such that complete lysis would give an absorbance reading at 410 nm wavelength of 1.0 after 1 hour of incubation at 37° C. The experiment was performed by adding GML (1 μg/ml) or phosphate buffered saline (PBS) to the RBCs, incubating for 1 hour, and then, after centrifugation to remove intact RBCs, the absorbance of the supernatant was determined. The data are summarized in Table 5. The absorbance at 410 nm of samples of RBCs alone or RBCs treated with GML was zero. Treatment of RBCs with α-hemolysin resulted in complete cell lysis. Incubation of RBCs with hemolysin and GML (1 μg/ml) completely prevented the lysis of RBCs.

TABLE 5
GML inhibits lysis of rabbit RBCs by Staphylococcal
exotoxin α-hemolysin.

| Experimental Group | Absorbance at 410 nm |
|---|---|
| RBC only | 0 |
| RBC + GML (1 μg/ml) | 0 |
| RBC + 0.2 μg α-hemolysin | 0.97 |
| RBC + 0.2 μg α-hemolysin + GML (1 μg/ml) | 0 |

### Example 7

### GML Inhibits the Cellular Lysis of RBCs by Bacillus anthracis Exotoxin

Experiments similar to those described in Example 6 were done to assess whether the B. anthracis exotoxin supernatants cause RBC lysis and whether such lysis can be inhibited by GML. Cultures of B. anthracis Step me were grown under conditions that favored production of exotoxins, including protective antigen, lethal factor, edema factor, and hemolysins. Sterne strain microorganisms were cultured with gentle shaking at 37° C. in 7% carbon dioxide in R medium and bicarbonate until they reached stationary phase. The supernatant from these cultures was able to lyse rabbit RBCs (Table 6) or human RBCs (Table 7). 0.1 ml aliquots of supernatant were added to 0.9 ml PBS containing rabbit or human RBCs. In the experiment with rabbit RBCs the samples were incubated at 37° C. for 15 minutes in 7% carbon dioxide, and in the experiment with human RBCs the samples were incubated for 18 hours under similar conditions. Controls contained R medium that had not been used to grow microbes. GML alone (at a concentration of 1, 5, or 10 μg/ml) did not cause RBC lysis. GML (at a concentration of 5 μg/ml) partially inhibited the lysis of rabbit RBCs by the B. anthracis exotoxin and almost completely inhibited the lysis at a concentration of 10 μg/ml (Table 6). GML (at a concentration of 1 μg/ml) partially inhibited the lysis of human RBCs by the B. anthracis exotoxin and completely inhibited the lysis at a concentration of 10 μg/ml (Table 7).

TABLE 6
GML prevents lysis of rabbit RBCs by B. anthracis toxin.

| Treatment Group | Absorbance 410 nm afler 15 minutes |
|---|---|
| RBC in PBS | 0 |
| RBG in water | 2.4 |
| RBC + 100 μl B. anthracis | 2.4 |
| supernatant | |
| RBC + 1 μg/ml GML | 0 |
| RBC + 5 μg/ml GML | 0 |
| RBC + 10 μg/ml GML | 0 |
| RBC + 100 μl B. anthracis | 2.4 |
| supernatant + 1 μg/ml GML | |
| RBC + 100 μl B. anthracis | 1.4 |
| supernatant + 5 μg/ml GML | |
| RBC + 100 μl B. anthracis | 0.02 |
| supernatant + 10 μg/ml GML | |

TABLE 7
GML prevents lysis of human RBCs by B. anthracis toxin.

| | | GML | GML | Absorbance |
|---|---|---|---|---|
| Treatment | Human RBCs | 1 μg/ml | 10 μg/ml | 410 nm |
| Water | + | − | − | 2.8 |
| PBS | + | + | − | 0 |
| PBS | + | − | + | 0 |
| Supernatant | + | − | − | 2.8 |
| Supernatant | + | + | − | 2.1 |
| Supernatant | + | − | + | 0 |

### Example 8

### GML Inhibits Streptolysin O and Streptolysin S-induced Hemolysis of RBCs

Group A Streptococci make two hemolysins, designated streptolysin O (oxygen labile and related to pneumolysin, listeriolysin, and numerous other hemolysins) and streptolysin S (oxygen stable). Streptolysin O is a heptamer pore-forming toxin whereas streptolysin S acts either as a surfactant that solubilizes the RBC cell membrane or as a protein that forms pores in the RBC cell membrane.

GML (10 μg/ml) was incorporated into culture plates containing solid agar and rabbit RBCs suspended in the agar. The effect of GML on. streptolysin O-induced hemolysis was tested by stabbing the Group A Streptococci into the agar so as to permit the anaerobic growth of the bacteria beneath the surface of the agar and thereby allow streptolysin O to function. Culture of Group A Streptococci on RBC-containing agar plates supplemented with GML (10 μg/ml) completely prevented hemolysis of RBCs.

The effect of GML on streptolysin S-induced hemolysis was assayed by direct culturing of Streptococci on the surface of the RBC-containing agar plates. When Group A Streptococci were cultured on the rabbit blood agar plates containing GML, streptolysin S-induced hemolysis was completely inhibited.

### Example 9

### GML is Non-Toxic to HVECs and Blocks HVEC Cytokine and Chemokine Release Following Stimulation with TSST-1 and Other Superantigens (SAgs)

### Generation and Culture of Immortal HVECs

One of the most important physical barriers to SAgs in humans is the intact epithelium. The inventor's recent studies have shown that the SAg TSST-1 up-regulates chemokine and cytokine gene expression in epithelial cells. An immortalized line of HVECs was obtained by transforming primary vaginal epithelial cells from a premenopausal woman with the E6/E7 genes of human papilloma virus 16. Monolayers of the immortalized HVEC were prepared and characterized by use of cytokeratin-specific antibody staining and by assessing the presence of cellular tight junctions. A mixture of monoclonal antibodies (mAb) (mAb AE1 and mAb AE3) specific for human cytokeratins bound, as expected, to the HVECs (data not shown), indicating that the cells were epithelial in nature. The AE1 mAb is specific for the high molecular weight (mw) cytokeratins 10, 14, 15, and 16 and the low mw cytokeratin 19 and the AE3 mAb is specific for the high molecular weight cytokeratins 1-6 and the low molecular weight cytokeratins 7 and 8. The cells formed partial tight junctions, consistent with the fact that vaginal epithelial cells, like oral epithelial cells but in contrast to epithelial cells of the intestinal tract, do not form tight junctions but form a permeability barrier by piling on top of one another and secreting water insoluble compounds such as ceramides, glucosyl ceramides, and cholesterol. The HVECs also had morphology typical of non-stratified squamous epithelial cells when grown in KSFM at 37° C. in 7% CO₂.

### Effect of S. aureus Exotoxin TSST-1 on the Cellular Morphology of HVECs

HVECs were examined for gross morphological effects by confocal microscopy following treatment with purified TSST-1. HVECs lost cell-to-cell contact and contracted following 6 hours of exposure to TSST-1 (100 μg/ml).

TSST-1 concentrations of 100 μg/ml are physiologically relevant to TSS S. aureus strains since, when cultured as thin films on tampons placed in dialysis tubing and then submerged beneath Todd Hewitt soft agar, TSS S. aureus produced 1.0-1.5 mg/ml of TSST-1. In addition, recent studies of TSST-1 production by methicillin resistant Staphylococcus aureus (NSA) isolates from TSS patients indicated that 100 to 1000 μg/ml of TSST-1 is produced by these strains when grown as thin films on polyethylene mesh. Thus, it is probable that TSS S. aureus growing on mucosal surfaces as thin films in vivo produce TSST-1 in excess of 100 μg/ml.

### S. aureus Exotoxin TSST-1 Enhances the Levels of Cytokine and Chemokine Expression in HVECs

The studies described below demonstrate that bacterial exotoxins up-regulate the expression of cytokines and chemokines in HVECs. The global responses (in terms of mRNA transcript production) of HVECs to TSST-1 (100 μg/ml) and untreated control HVECs following culture for 3 and 6 hours were determined using the Affymetrix Human GeneChip® U133A. Treatment of HVECs for 3 and 6 hours with TSST-1 (100 μg/ml) caused significant up- and down-regulation (by two fold or greater) of the expression of 1472 genes and 2386 gene, respectively. As shown in Table 8, chemokine genes whose transcript levels were significantly up-regulated by 6 hours included, for example, CCL20 (encoding MIP-3α; 169-fold), CXCL1 (encoding GRO-α; 84 fold), CXCL2 (encoding GRO-β; 13 fold), and CXCL3 (encoding GRO-γ, 32 fold). In addition, genes encoding cytokines whose transcript levels were also significantly up-regulated included, for example, those encoding TNF-α and IL-1β, with changes of 2.5-fold and 2.0-fold, respectively. Moreover, mRNA expression of major histocompatibility complex (MHC) class I classical genes (A, B, C) and non-classical genes (E, F, and G) was significantly up-regulated in response to TSST-1 (100 ug/ml) (data not shown).

TABLE 8
Genes in HVECs whose expression was up-regulated after
exposure to TSST-1 for 3 or 6 hours.

| | Fold Change | Fold Change |
|---|---|---|
| Gene Name | TSST-1 (3 h) | TSST-1 (6 h) |
| CCL20 (MIP-3α) | NS^{a} | 169 |
| CXCL1 (GRO-α) | 16 | 84.0 |
| CXCL2 (GRO-β) | 4.9 | 13.0 |
| CXCL3 (GRO-γ) | 4 | 32.0 |
| CXCL14 | 12 | 5.0 |
| Interleukin 1-β | NS | 2.0 |
| Interleukin-6 | NS | 2.3 |
| Interleukin-8 | 8 | 48.5 |
| Interleukin-15 | 4.3 | 9.8 |
| TNF-α | NS | 2.5 |

^{a}NS, No significant change from control

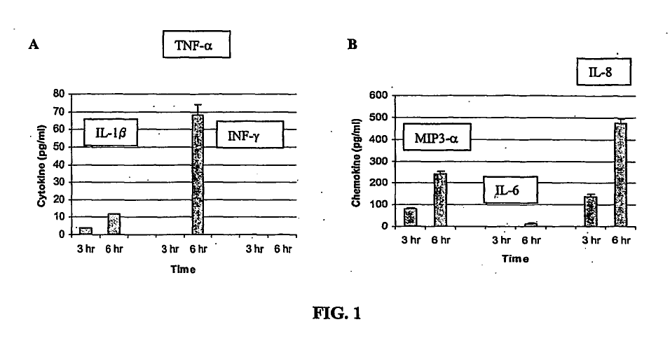

Representative cytokine and chemokine genes whose expression was determined by the above-described microarray analysis to be up-regulated following exposure of HVECs to TSST-1 (100 μg/ml) were analyzed in terms of encoded protein production using Enzyme-Linked Immunosorbent Assays (ELISAs). Specifically, cytokine (IL-1β, TNF-α, and interferon γ) and chemokine (MP-3α, IL-6, and IL-8) concentrations were determined in the supernatants of cultured HVECs incubated with TSST-1 (100 μg/ml) for 3 or 6 hours (FIG. 1). Ovalbumin (100 μg/ml) (rather than TSST-1) was added to control cultures and was found to cause only minimal production of cytokines and chemokines from the HVECs after the 6 hours of culture (see Table 9), thereby indicating that the effects seen with TSST-1 (100 μg/ml) were caused by the exotoxin itself and were not non-specific effects that would be elicited by any exogenous protein.

Significant levels of both IL-1β and TNF-α were detected in culture supernatants following incubation of HVECs with TSST-1 (100 μg/ml) for 6 hours (12 pg/ml and 68 pg/ml, respectively) (FIG. 1). As shown in Table 7, the levels of IL-1β and TNF-α proteins were consistent with the measured transcriptional induction of these genes (2.0 and 2.5-fold, respectively) by TSST-1. In contrast, but consistent with the inability to detect interferon-γ transcripts, interferon-γ protein was not detected in culture supernatants following incubation of HVECs with TSST-1 (100 μg/ml).

The chemokines MIP-3α (240 pg/ml), IL-6 (15 pg/ml), and IL-8 (475 pg/ml) were detected in culture supernatants following incubation of HVECs with TSST-1 (100 μg/ml) for 6 hours. Control HVECs (without TSST-1) did not produce detectable levels of the cytokines or chemokines tested for after 3 and 6 hours of incubation.

HVECs were cultured as described above in the presence of various SAgs, with or without GML. As shown in Table 9, GML was highly effective in inhibiting SAg-induced production of two chemokines (MIP-3α and IL-8) by HVECs after 6 hours of culture with the following SAgs (100 μg/ml): TSST-1, Staphylococcal enterotoxin B (SEB), and Streptococcal pyrogenic exotoxin A (SPEA). That GML did not interfere with protein detection in the ELISAs was shown by the fact that it did not affect chemokine detection when added to control supernatants containing the relevant chemokines. The lower limit of detection for all cytokines and chemokines was 4 pg/ml to 16 pg/ml. Collectively, these data show that GML inhibits inflammatory responses in HVECs induced by SAgs.

TABLE 9
GML inhibits of chemokines and cytokine secretion by HVECs
in response to TSST-1 and others SAgs.

| Protein Tested | | MIP-3α (pg/ml ± | |
|---|---|---|---|
| (100 μg/ml) | GML (20 μg/ml) | SE) | IL-8 (pg/ml ± SE) |
| None | − | 9.0 ± 1.5 | 16 ± 1.8 |
| None | + | None Detected | None Detected |
| Ovalbumin | − | 12 ± 1.8 | 36 ± 1.5 |
| TSST-1 | − | 320 ± 4 | 500 ± 33 |
| TSST-1 | + | None Detected | None Detected |
| SEB | − | 470 ± 7 | 620 ± 7 |
| SEB | + | None Detected | None Detected |
| SPEA | − | 396 ± 8 | 470 ± 10 |
| SPEA | + | None Detected | None Detected |

### Example 10

### GML Inhibits HIV-1 Infection of Human Peripheral Blood Mononuclear Cells (PBMC)

A culture of human PBMC isolated from an HIV seronegative donor was divided into three samples. All three samples were cultured with the Superantigen Staphylococcal Enterotoxin A (SEA; at a final concentration of 0.1 μg/ml) and recombinant human IL-2 (rIL-2; final concentration of 10 units/ml) for 48 hours and then infected with HIV-1 (at a final concentration of 100 tissue culture infectious doses (TCID)₅₀/ml). GML (at a final concentration of 100 μg/ml) was added to one of the samples immediately before and two days after HIV-1 infection and to the second sample at the same time as the HIV-1 infection. No GML was added to the third control sample. All three samples were then cultured for 5 days. Cells were harvested from the three samples and spotted onto glass microscope slides, fixed, and immunochemically stained with antibody specific for HIV-1 p24 protein. The majority of the cells in the sample not containing GML expressed p24. On the other hand the sample treated before and after HIV-1 infection and the sample treated with GML simultaneous with HIV-1 infection contained rare and no cells expressing p24, respectively. Cell viability was preserved in the GML-containing samples but the total number of cells in the samples was significantly less than in the control sample not containing GML. The latter finding is consistent with the inhibition by GML of SEA- and IL-2-activated cell proliferation.

A number of embodiments of the invention have been described. Nevertheless, it will be understood that various modifications may be made without departing from the spirit and scope of the invention. Accordingly, other embodiments are within the scope of the following claims.

What is claimed is:

1. A method of inhibiting a cell membrane-mediated effect, the method comprising:
(a) identifying a vertebrate subject as having been, as likely to have been, or as likely to be, exposed to an infectious microorganism, wherein the infectious microorganism, or a microbial factor associated with the infectious microorganism, has a cell membrane-mediated effect on a vertebrate cell, the cell membrane-mediated effect being associated with a pathological condition of the vertebrate subject; and
(b) administering to the vertebrate subject an isolated glycerol-based compound that (i) inhibits the cell membrane-mediated effect and (ii) comprises a structure selected from the group consisting of: 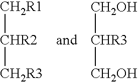
wherein R1 is: OH; CO(CH₂)₈CH₃; CO(CH₂)₉CH₃; CO(CH₂)₁₀CH₃; CO(CH₂)₁₁CH₃: CO(CH₂)₁₂CH₃; O(CH₂)₉CH₃; O(CH₂)10CH₃; O(CH₂)₁₁CH₃; O(CH₂)₁₂CH₃; or O(CH₂)₁₃CH₃,
R2 is: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃, and
R3 is: CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃.

2. The method of claim 1, wherein the cell membrane-mediated effect comprises facilitation of entry of the infectious microorganism into the cell.

3. The method of claim 1, wherein the cell membrane-mediated effect results in activation of the vertebrate cell.

4. The method of claim 3, wherein activation of the vertebrate cell increases synthesis or secretion by the vertebrate cell of mediators that enhance inflammation in the subject or enhance infection of the subject by the infectious microorganism.

5. The method of claim 1, wherein the cell membrane-mediated effect results in lysis of the vertebrate cell.

6. The method of claim 1, wherein the glycerol-based compound inhibits the cell membrane-mediated effect by the induction of apoptosis in the vertebrate cell.

7. The method of claim 1, wherein the glycerol-based compound inhibits the cell membrane-mediated effect caused by the induction of cellular anergy in the vertebrate cell.

8. The method of claim 1, wherein the vertebrate cell is an epithelial cell.

9. The method of claim 8, wherein the epithelial cell is a vaginal cell.

10. The method of claim 1, wherein the vertebrate cell is a red blood cell (RBC).

11. The method of claim 1, wherein the vertebrate cell is a lymphocyte.

12. The method of claim 11, wherein the lymphocyte is a T cell.

13. The method of claim 11, wherein the lymphocyte is a B cell.

14. The method of claim 1, wherein the infectious microorganism is a bacterium.

15. The method of claim 14, wherein the bacterium is Staphylococcus.

16. The method of claim 15, wherein the Staphylococcus is Staphylococcus aureus.

17. The method of claim 14, wherein the bacterium is Neisseria.

18. The method of claim 17, wherein the Neisseria is Neisseria gonorrheae.

19. The method of claim 14, wherein the bacterium is a Streptococcus.

20. The method of claim 19, wherein the Streptococcus is Streptococcus pyogenes.

21. The method of claim 14, wherein the bacterium is Bacillus.

22. The method of claim 21, wherein the bacterium is Bacillus anthracis.

23. The method of claim 14, wherein the bacterium is Clostridium.

24. The method of claim 23, wherein the bacterium is Clostridium perfringens.

25. The method of claim 14, wherein the bacterium is Chlamydia trachomatis.

26. The method of claim 14, wherein the bacterium is selected from the group consisting of Gardnerella vaginalis, Haemophilus ducreyi, and Treponema pallidum.

27. The method of claim 1, wherein the infectious microorganism is a virus.

28. The method of claim 27, wherein the virus is Herpes Simplex Virus (HSV).

29. The method of claim 27, wherein the virus is Human Papilloma Virus (HPV).

30. The method of claim 1, wherein the infectious microorganism is a protozoan.

31. The method of claim 30, wherein the protozoan is Trichomonas vaginalis.

32. The method of claim 1, wherein the infectious microorganism is a fungus.

33. The method of claim 32, wherein the fungus is Candida albicans.

34. The method of claim 1, wherein the subject is human.

35. A method of inhibiting a cell-membrane mediated effect, the method comprising:
(a) identifying a vertebrate subject as having been, as likely to have been, or likely to be exposed to a factor that has cell-mediated effect on a vertebrate cell, wherein the cell-mediated effect can result in a pathologic condition in the vertebrate subject; and
(b) administering to the subject an isolated glycerol based compound that (i) inhibits the cell membrane-mediated effect on the vertebrate cell by the factor and (ii) comprises a structure selected from the group consisting of: 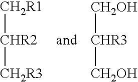
wherein R1 is: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃,
R2 is: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃, and
R3 is: CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)10CH₃; O(CH₂)₁₁CH₃; O(CH₂)₁₂CH₃; or O(CH₂)₁₃CH₃.

36. The method of claim 35, wherein the factor is a microbial factor associated with an infectious microorganism.

37. The method of claim 36, wherein the cell membrane-mediated effect comprises facilitation of entry of the infectious microorganism into the vertebrate cell.

38. The method of claim 35, wherein the cell membrane-mediated effect results in activation of the vertebrate cell.

39. The method of claim 38, wherein activation of the vertebrate cell increases synthesis or secretion by the vertebrate cell of mediators that enhance inflammation in the subject or enhance infection of the subject by the infectious microorganism.

40. The method of claim 35, wherein the cell membrane-mediated effect results in lysis of the vertebrate cell.

41. The method of claim 35, wherein the glycerol-based compound inhibits the cell membrane-mediated effect by the induction of apoptosis in the vertebrate cell.

42. The method of claim 35, wherein the glycerol-based compound inhibits the cell membrane-mediated effect by the induction of cellular anergy in the vertebrate cell.

43. The method of claim 35, wherein the factor is an exotoxin.

44. The method of claim 43, wherein the exotoxin is a hemolysin.

45. The method of claim 43, wherein the exotoxin is a superantigen.

46. The method of claim 44, wherein the hemolysin is selected from the group consisting of: α-hemolysin, streptolysin O, streptolysin S, pneumolysin, listerolysin, perfringolysin, and Bacillus anthracis hemolysin.

47. The method of claim 43, wherein the exotoxin is selected from the group consisting of toxic shock syndrome toxin-1, Streptococcal pyrogenic exotoxin, Staphylococcal enterotoxin, A-B toxins, Diptheria exotoxin, Cholera exotoxin, Pertussis exotoxin, Shiga toxin, Shiga-like toxin, anthrax toxin, Botulinal exotoxin, Tetanus exotoxin, tracheal cytotoxin, Helicobacter toxins, alpha toxin from Clostridium perfringens (lecithinase), kappa toxin (collagenase), mu toxin (hyaluronidase), leukocidin, elastase, and Staphylococcal α-hemolysin.

48. The method of claim 35, wherein the vertebrate cell is an epithelial cell.

49. The method of claim 48, wherein the epithelial cell is a vaginal cell.

50. The method of claim 35, wherein the vertebrate cell is an RBC.

51. The method of claim 35, wherein the vertebrate cell is a lymphocyte.

52. The method of claim 51, wherein the lymphocyte is a T cell.

53. The method of claim 51, wherein the lymphocyte is a B cell.

54. The method of claim 35, wherein the pathological condition is psoriasis.

55. The method of claim 35, wherein the pathological condition is atopic dermatitis.

56. The method of claim 35, wherein the pathological condition comprises skin papules or pustules.

57. The method of claim 35, wherein the pathological condition is selected is selected from the group consisting of: toxic shock syndrome, pneumonia, bacteremia in association with cutaneous infection, deep soft tissue infection, meningitis, peritonitis, osteomyelitis, septic arthritis, postpartum sepsis, neonatal sepsis, endotoxemias, and exotoxemias, and food poisoning.

58. An in vitro method of inhibiting a cell-membrane-mediated effect, the method comprising:
(a) culturing a vertebrate cell with (i) an infectious microorganism that has a cell membrane-mediated effect on the vertebrate cell, or that elicits the production in the culture of a vertebrate mediator that has the cell membrane-mediated effect on the vertebrate cell; or (ii) a microbial factor that causes the cell membrane-mediated effect on the vertebrate cell; and
(b) before, simultaneous with, or after step (a), contacting the vertebrate cell with an isolated glycerol-based compound that (i) inhibits the cell membrane-mediated effect on vertebrate cells and (ii) comprises a structure selected from the group consisting of: 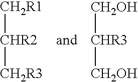
wherein R1 is: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃,
R2 is: OH; CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃, and
R3 is: CO(CH₂)₈CH₃; CO(CH₂)₉CH₃; CO(CH₂)₁₀CH₃; CO(CH₂)₁₁CH₃; CO(CH₂)₁₂CH₃; O(CH₂)₉CH₃; O(CH₂)₁₀CH₃; O(CH₂)₁₁CH₃; O(CH₂)₁₂CH₃; or O(CH₂)₁₃CH₃.

59. The method of claim 58, wherein the cell membrane-mediated effect results in activation of the vertebrate cell.

60. The method of claim 58, wherein the cell membrane-mediated effect comprises facilitation of entry of an infectious microorganism into the vertebrate cell.

61. The method of claim 59, wherein activation of the vertebrate cell increases synthesis or secretion by the vertebrate cell of mediators that enhance inflammation in the subject or enhance infection of the subject by the infectious microorganism.

62. The method of claim 58, wherein the cell membrane-mediated effect results in lysis of the vertebrate cell.

63. The method of claim 58, wherein the glycerol-based compound inhibits the cell membrane-mediated effect by the induction of apoptosis in the vertebrate cell.

64. The method of claim 58, wherein the glycerol-based compound inhibits the cell membrane-mediated effect by the induction of cellular anergy in the vertebrate cell.

65. The method of claim 58, wherein the factor is an exotoxin.

66. The method of claim 65, wherein the exotoxin is a hemolysin.

67. The method of claim 65, wherein the exotoxin is a superantigen.

68. The method of claim 66, wherein the hemolysin is selected from the group consisting of: α-hemolysin, streptolysin O, streptolysin S, pneumolysin, listerolysin, perfringolysin, and Bacillus anthracis hemolysin.

69. The method of claim 65, wherein the exotoxin is selected from the group consisting of toxic shock syndrome toxin-1, Streptococcal pyrogenic exotoxin, Staphylococcal enterotoxins, A-B toxins, Diptheria exotoxin, Cholera exotoxin, Pertussis exotoxin, Shiga toxin, Shiga-like toxin, anthrax toxin, Botulinal exotoxin, Tetanus exotoxin, tracheal cytotoxin, Helicobacter toxin, alpha toxin (lecithinase), kappa toxin (collagenase), mu toxin (hyaluronidase), leukocidin, elastase, and Staphylococcal α-hemolysin.

70. The method of claim 58, wherein the vertebrate cell is an epithelial cell.

71. The method of claim 70, wherein the epithelial cell is a vaginal cell.

72. The method of claim 58, wherein the vertebrate cell is an RBC.

73. The method of claim 58, wherein the vertebrate cell is a lymphocyte.

74. The method of claim 73, wherein the lymphocyte is a T cell.

75. The method of claim 73, wherein the lymphocyte is a B cell.

76. The method of claim 58, wherein the infectious microorganism is a bacterium.

77. The method of claim 76, wherein the bacterium is a Staphylococcus.

78. The method of claim 77, wherein the Staphylococcus is Staphylococcus aureus.

79. The method of claim 76, wherein the bacterium is a Neisseria.

80. The method of claim 79, wherein the Neisseria is Neisseria gonorrheae.

81. The method of claim 76, wherein the bacterium is a Streptococcus.

82. The method of claim 81, wherein the Streptococcus is Streptococcus pyogenes.

83. The method of claim 76, wherein the bacterium is Bacillus.

84. The method of claim 83, wherein the Bacillus is Bacillus anthracis.

85. The method of claim 76, wherein the bacterium is Clostridium.

86. The method of claim 85, wherein the Clostridium is Clostridium perfringens.

87. The method of claim 76, wherein the bacterium is Chlamydia trachomatis.

88. The method of claim 76, wherein the bacterium is selected from the group consisting of Gardnerella vaginalis, Haemophilus ducreyi, and Treponema pallidum.

89. The method of claim 58, wherein the infectious microorganism is a virus.

90. The method of claim 89, wherein the virus is Herpes Simplex Virus (HSV).

91. The method of claim 89, wherein the virus is Human Papilloma Virus (HPV).

92. The method of claim 58, wherein the infectious microorganism is a protozoan.

93. The method of claim 92, wherein the protozoan is Trichomonas vaginalis.

94. The method of claim 58, wherein the infectious microorganism is a fungus.

95. The method of claim 94, wherein the fungus is Candida albicans.

96. The method of claim 58, wherein the subject is a human.
